(19) United States
(12) Patent Application Publication
(10) Pub. No.: US 2024/0390469 A1
(43) Pub. Date: Nov. 28, 2024

(54) TARGETED TRADITIONAL CHINESE MEDICINE IN-SITU TUMOR VACCINE, AND PREPARATION METHOD AND APPLICATION THEREOF

(71) Applicant: NANJING UNIVERSITY OF CHINESE MEDICINE, Nanjing (CN)

(72) Inventors: Jinao DUAN, Nanjing (CN); Jialun DUAN, Nanjing (CN); Chunjie BAO, Nanjing (CN); Liqiang ZHAO, Nanjing (CN); Sheng GUO, Nanjing (CN)

(73) Assignee: NANJING UNIVERSITY OF CHINESE MEDICINE, Nanjing (CN)

(21) Appl. No.: 18/795,981

(22) Filed: Aug. 6, 2024

(30) Foreign Application Priority Data
May 11, 2024 (CN) 2024105796711

(51) Int. Cl. A61K 39/00; A61K 9/51; A61K 47/10; A61K 47/26; A61P 35/00

(52) U.S. Cl. CPC A61K 39/0013; A61K 9/5153; A61K 9/5176; A61K 9/5192; A61K 47/10; A61K 47/26; A61P 35/00; A61K 2039/55555; A61K 2039/55566; A61K 2039/852

(57) ABSTRACT

Disclosed are a targeted traditional Chinese medicine in-situ tumor vaccine, a preparation method thereof and an application thereof in resisting pancreatic cancer. In the invention, PLGA nanospheres are used to carry Lycium barbarum polysaccharide and Brusatol, and subjected to surface modification with a macrophage membrane and a c-RGD peptide. The invention can be targeted at a tumor site in vivo, so that the tumor undergoes immunogenic cell death and releases tumor antigens, and can also regulate immune cells at the same time, so as to remodel the tumor microenvironment, and achieve robust anti-pancreatic cancer effect. In the invention, the preparation method is simple, and the tumor vaccine is suitable for carrying a cancer therapeutic drug, especially a drug for treating pancreatic cancer.

### CROSS-REFERENCE TO RELATED APPLICATIONS

This application claims priority to Chinese Patent Application Ser. No. CN2024105796711 filed on 11 May 2024.

### TECHNICAL FIELD

The present invention belongs to the technical field of biomedical materials, and particularly relates to a targeted traditional Chinese medicine in-situ tumor vaccine, a preparation method thereof and an application thereof in resisting pancreatic cancer.

### BACKGROUND

Pancreatic cancer is a common malignant tumor of digestive tract, and has the characteristics of being difficult to find and treat and easy to cause death. Traditional surgical therapy, radiation therapy (radiotherapy) and chemical therapy (chemotherapy) have limited effects on the pancreatic cancer, and the lack of effective treatment means leads to poor prognosis of pancreatic cancer. In recent years, immunotherapy has been gradually applied to the treatment of various types of tumors, but the application in clinical trial of pancreatic cancer has not achieved an expected curative effect. One of the main reasons for the failure of immunotherapy is that a tumor microenvironment (TME) of the pancreatic cancer has the defect of lacking infiltration by high-quality effector T cells (low or weak immunogenicity of the pancreatic cancer) and immunosuppressive cells (such as M2 macrophages and Treg cells) in an immunosuppressive tumor microenvironment, which leads to the inherent immune escape of tumor. At present, drugs for treating the pancreatic cancer are mainly tabine drugs, platinum drugs, pyrimidine drugs, and the like, but these drugs have the characteristics of poor targeting ability and great toxic and side effects, which lead to a poor therapeutic effect and a severe harm to human body.

There is a new application of drug-carrying nanospheres based on surface modification by a biological membrane in a drug delivery system, wherein the drug-carrying nanospheres processed on the basis of a natural cell membrane, which are represented by the natural cell membrane, and natural extracellular vesicles (EVs) prepared from an exosome are widely applied. In recent years, the above two drug-carrying delivery systems based on the biological membrane material have played a great role in tumor immunotherapy, and have become the research focus of tumor targeted drug administration.

Macrophage is an immune cell with strong phagocytosis, and plays a key role in the induction and regulation of specific immune response. Macrophage membrane has been widely studied as the biological membrane material in the study of the drug-carrying delivery system based on the biological membrane. C-RGD peptide (cyclic-Arg-Gly-Asp) is a cyclic polypeptide composed of three amino acids, arginine-glycine-aspartic acid, which may competitively bind to an integrin receptor on a tumor surface, so as to achieve a tumor targeting effect. It has been proved by studies that nanospheres coated with the macrophage membrane, poly(lactic-co-glycolic acid) (PLGA for short), can effectively retain an antigenic exterior on a surface of an immune cell membrane, so as to maintain the immunogenicity of immune cells, thus playing a targeted delivery function in the treatment of tumor and inflammation.

However, at present, there is no study on the treatment of pancreatic cancer by embedding the therapeutic drug in a drug-carrying system of nanospheres wrapped by the macrophage membrane and the c-RGD peptide.

Therefore, it is very necessary to develop a new drug-carrying system capable of achieving accurate treatment of targeted pancreatic cancer.

### SUMMARY

Therefore, the technical problem to be solved by the present invention is that a traditional drug for treating pancreatic cancer has a poor targeting ability and a poor slow release performance, so as to provide a targeted delivery nanosphere drug based on an immune cell membrane with a good targeting ability and a slow release function, and a preparation method and application thereof.

In order to solve the above technical problem, the technical solutions of the present invention are as follows.

In a first aspect of the present invention, a targeted traditional Chinese medicine in-situ tumor vaccine is provided, which comprises a drug, PLGA nanospheres and a surface modification system, wherein the drug is embedded into the PLGA nanospheres, the surface modification system is a macrophage membrane and c-RGD peptide modification system, and the drug is Lycium barbarum polysaccharide and Brusatol. A mass ratio of the PLGA nanospheres to the macrophage membrane is 5:1 to 10:1, a mass ratio of the PLGA nanospheres to the c-RGD peptide is 15:1 to 20:1, a mass ratio of the PLGA nanospheres to the Lycium barbarum polysaccharide is 4:1 to 8:1, and a mass ratio of the PLGA nanospheres to the Brusatol is 20:1 to 30:1.

As a preferred solution, the macrophage membrane is extracted by polarizing RAW264.7 cell line.

As a preferred solution, an average particle size of the targeted traditional Chinese medicine in-situ tumor vaccine wrapped by the macrophage membrane and the c-RGD peptide is less than 200 nm.

In a second aspect of the present invention, a preparation method of the targeted traditional Chinese medicine in-situ tumor vaccine is provided, which comprises the following steps:
S1: extracting the macrophage membrane: lysing macrophages by a cell lysis buffer, and then extracting the cell membrane by a layered high-speed centrifugation method for standby application;
S2: preparing the PLGA nanospheres in which the drug is embedded: embedding the drug into the PLGA nanospheres by a double emulsification method; and
S3: wrapping the PLGA nanospheres containing the drug by the surface modification system.

As a preferred solution, in the step S1, the macrophage membrane is obtained by polarizing RAW264.7 cell line, which comprises steps of: polarizing RAW264.7 into M1-type macrophages by lipopolysaccharide; centrifugally collecting the M1-type macrophages, and washing the collected M1-type macrophages with PBS twice; and extracting the macrophage membrane by a cell membrane protein and cytoplasm protein extraction kit (Beyotime, P0033).

As a preferred solution, in the step S3, the PLGA nanospheres containing the drug are wrapped by the surface modification system by a membrane extrusion method, wherein the membrane extrusion method comprises: mixing the surface modification system with the PLGA nanospheres containing the drug, and then subjecting the obtained mixture to an extrusion treatment by an extruder, wherein a polycarbonate membrane is used as a filter structure in a process of the extrusion treatment.

As a preferred solution, in the step S2, the Lycium barbarum polysaccharide and the Brusatol are embedded into the PLGA nanospheres by the double emulsification method, wherein the double emulsification method comprises: dissolving the PLGA in an acetone organic solvent to obtain a premix; dissolving the Brusatol in dimethyl sulfoxide, dissolving the Lycium barbarum polysaccharide in normal saline, adding the solutions into the premix under stirring, and subjecting the mixture to an ice bath ultrasonic treatment to form a water-oil emulsion; uniformly mixing the water-oil emulsion with a solution containing poloxamer 188 to obtain a water-oil-water emulsion; and subjecting the water-oil-water emulsion to an ice bath ultrasonic treatment or a homogenization treatment, then removing excessive acetone by volatilization, ultra-freezing, centrifuging and washing the emulsion, and then freeze-drying the emulsion to obtain the PLGA nanospheres in which drug molecules are embedded.

As a preferred solution, before freeze-drying, the method further comprises a step of adding a freeze-drying protective agent, wherein the freeze-drying protective agent is polyvinyl alcohol or mannitol, and a mass concentration of the freeze-drying protective agent is 0.5% to 1% (w/v).

In the present invention, an application of the targeted traditional Chinese medicine in-situ tumor vaccine in preparing a cancer therapeutic drug is provided.

Compared with the prior art, the above technical solutions of the present invention have the following advantages:

The targeted traditional Chinese medicine in-situ tumor vaccine provided by the present invention comprises the targeted traditional Chinese medicine in-situ tumor vaccine caoted by the macrophage membrane and the c-RGD peptide, and comprises the drug, the PLGA nanospheres and the surface modification system, wherein the drug is coated into the PLGA nanospheres, the surface modification system is the macrophage membrane and c-RGD peptide modification system, and the drug is the Lycium barbarum polysaccharide and the Brusatol. The mass ratio of the PLGA nanospheres to the macrophage membrane is 5:1 to 10:1, the mass ratio of the PLGA nanospheres to the c-RGD peptide is 15:1 to 20:1, the mass ratio of the PLGA nanospheres to the Lycium barbarum polysaccharide is 4:1 to 8:1, and the mass ratio of the PLGA nanospheres to the Brusatol is 20:1 to 30:1. The targeted traditional Chinese medicine in-situ tumor vaccine may be targeted at a tumor site in vivo, and the slow-released drug can regulate a function of immune cells and cause immunogenic death of pancreatic cancer cells, so as to trigger an inflammatory reaction at the tumor site, and achieve robust treatment of pancreatic cancer, and the preparation method of the targeted traditional Chinese medicine in-situ tumor vaccine is simple, and the tumor vaccine is suitable for carrying a cancer therapeutic drug, especially a drug for treating pancreatic cancer.

### BRIEF DESCRIPTION OF THE DRAWINGS

FIG. 1 is a schematic diagram of preparation and a targeting effect of a targeted traditional Chinese medicine in-situ tumor vaccine in the present invention;

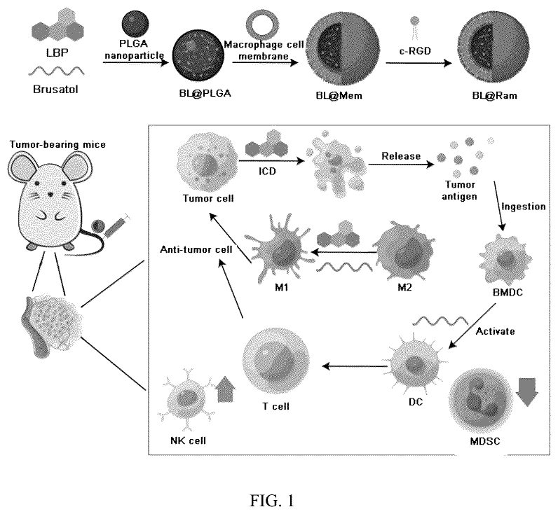

FIG. 2 shows activation efficacy of refined Lycium barbarum polysaccharides with different molecular weights on BMDC cells;

FIG. 3a shows the dispersion effect detection results of the targeted traditional Chinese medicine in-situ tumor vaccine in the present invention;

FIG. 3b shows the particle size detection results of the targeted traditional Chinese medicine in-situ tumor vaccine in the present invention;

FIG. 3c shows the Zeta potential detection results of the targeted traditional Chinese medicine in-situ tumor vaccine in the present invention;

FIG. 3d shows release rates of Lycium barbarum polysaccharide in the vaccine in solutions at different pH values;

FIG. 3e shows release rates of Brusatol in the vaccine in solutions at different pH values;

FIG. 4a shows the characterization of related representations of the targeted traditional Chinese medicine in-situ tumor vaccine in the present invention (transmission electron microscope (TEM) images of the tumor vaccine wrapped by the c-rgd and the macrophage membrane);

FIG. 4b shows the characterization of related representations of the targeted traditional Chinese medicine in-situ tumor vaccine in the present invention (transmission electron microscope (TEM) images of the tumor vaccine wrapped by the macrophage membrane);

FIG. 4c shows the characterization of related representations of the targeted traditional Chinese medicine in-situ tumor vaccine in the present invention (transmission electron microscope (TEM) images of the tumor vaccine without wrapping by the c-rgd and the macrophage membrane);

FIG. 4d shows the characterization of related representations of the targeted traditional Chinese medicine in-situ tumor vaccine in the present invention (SDS-PAGE gel);

FIG. 5 is a graph of cellular uptake of the targeted traditional Chinese medicine in-situ tumor vaccine by flow cytometry in the present invention;

FIG. 6 shows diagrams of an activation effect of the targeted traditional Chinese medicine in-situ tumor vaccine on dendritic cells (wherein the brown diagram is a control group, the blue diagram is a targeted traditional Chinese medicine in-situ tumor vaccine group only added with the Brusatol (B@Ram for short), and the red diagram is a targeted traditional Chinese medicine in-situ tumor vaccine group (BL@Ram for short));

FIG. 7 is a diagram of a therapeutic effect of the targeted traditional Chinese medicine in-situ tumor vaccine in the present invention on tumor-bearing mice with pancreatic cancer.

### DETAILED DESCRIPTION

The present invention is further described hereinafter with reference to the drawings and specific embodiments, so that those skilled in the art can better understand and implement the present invention. However, the mentioned embodiments should not be taken as a limitation of the present invention.

Experimental materials of the following embodiments:

Preparation method of Lycium barbarum polysaccharide: 10 kg of dried fructus lycii was weighed and added into 80% ethanol according to a solid-liquid ratio of 10:1, heated, refluxed and extracted twice, 1 hour each time, so as to obtain an ethanol extracting solution and an ethanol extracted residue. Deionized water in a volume 15 times that of the ethanol extracted residue was added into the ethanol extracted residue, heated, refluxed and extracted twice, 1 hour each time, water extracting solutions were combined, added with anhydrous ethanol to make a volume fraction of ethanol reach 80%, allowed to stand at room temperature overnight, and centrifuged at 4500 rpm for 5 minutes, a precipitate was collected, washed with anhydrous ethanol, and freeze-dried, so as to obtain crude Lycium barbarum polysaccharide. After the crude Lycium barbarum polysaccharide was dissolved with deionized water, the crude Lycium barbarum polysaccharide solution was input into ultrafiltration equipment through a circulating pump, and ultrafiltered by a 100 kD cellulose membrane. An intercepting solution was circularly ultrafiltered, and the collected intercepting solution was a refined Lycium barbarum polysaccharide sample solution with a molecular weight greater than 100 kD. An ultrafiltration membrane permeating solution was crude Lycium barbarum polysaccharide with a molecular weight less than 100 kD. In the same way, the crude Lycium barbarum polysaccharide was separated by 50 kD and 3 kD ultrafiltration membrane assemblies in sequence, so as to obtain refined Lycium barbarum polysaccharide sample solutions of 50 kD to 100 kD, 3 kD to 50 kD and <3 kD. The refined Lycium barbarum polysaccharide with the molecular weight of 50 kD to 100 kD was used in the following embodiments.

The Brusatol was purchased from ChemFaces (14907-98-3), the PLGA (poly(lactic-co-glycolic acid) was purchased from Aladdin (P647488), and the c-RGD (cRGD-PEG-DSPE) was purchased from MeloPEG (310702).

### Embodiment 1 Immune Activation Situations of Refined Lycium barbarum Polysaccharides with Different Molecular Weights on Tumor-Bearing Mice

in this embodiment, immune synergism situations of Lycium barbarum polysaccharides with different molecular weights were screened, and verified as follows.

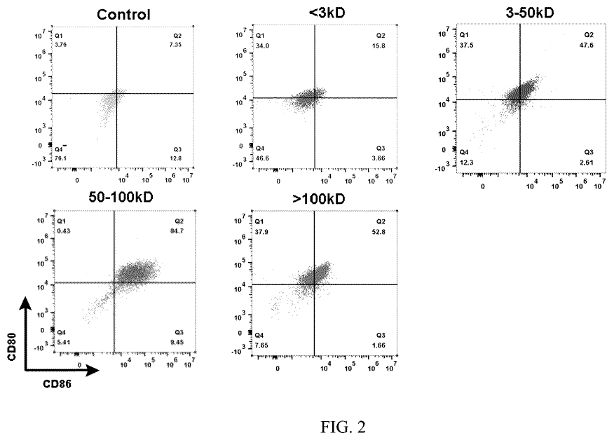

BMDCs were treated with Lycium barbarum polysaccharide solutions with different molecular weights (<3 kD, 3-50 kD, 50-100 kD and >100 kD). 2 days later, BMDCs were collected, and activation levels of BMDC were detected by flow cytometry. Results were shown in FIG. 2, proportions of CD80 and CD86 in BMDCs activated by refined Lycium barbarum polysaccharide of 50 kD to 100 kD were the highest, so that the refined Lycium barbarum polysaccharide of 50 kD to 100 kD was selected to prepared a targeted traditional Chinese medicine in-situ tumor vaccine.

### Embodiment 2 Preparation of Targeted Traditional Chinese Medicine In-Situ Tumor Vaccine

The targeted traditional Chinese medicine in-situ tumor vaccine provided by this embodiment was prepared by the following method.

In S1, a macrophage membrane was extracted: The macrophage membrane was extracted by polarizing RAW264.7, which comprised the step of polarizing RAW264.7 into M1-type macrophages by lipopolysaccharide.

The M1-type macrophages were centrifugally collected, and washed with PBS twice; and the macrophage membrane was extracted by a cell membrane protein and cytoplasm protein extraction kit (Beyotime, P0033).

In S2, PLGA nanospheres in which drugs were embedded were prepared: Drugs were embedded into the PLGA nanospheres by a double emulsification method.

In S21, 10 mg of the PLGA nanospheres were dissolved in 1 mL of acetone at room temperature to obtain a premix.

In S22, 2 mg of the Lycium barbarum polysaccharide was dissolved in 0.2 mL of normal saline, 0.3 mg of the Brusatol was dissolved in 0.08 mL of dimethyl sulfoxide, and the solutions were slowly dropwise added into the above premix under magnetic stirring at a stirring speed of 1500 rpm as an internal water phase.

The obtained liquid was subjected to an ice bath ultrasonic treatment (the ultrasonic power was set at 300 W, the ultrasonic device was turned on for 3 seconds and then turned off for 2 seconds, and the ultrasonic treatment lasted for a total of 2 minutes), so as to obtain a primary emulsion.

In S23, 2 mg of poloxamer 188 (0.1% w/v) was weighed and dissolved in 2 mL of normal saline as an external water phase (2 mL of the external water phase corresponded to 1 mL of the primary emulsion).

The primary emulsion was slowly dropwise added into the above external water phase under magnetic stirring at a stirring speed of 1500 rpm, so as to obtain a multiple emulsion.

The obtained multiple emulsion was placed in a fume hood to volatilize excess acetone, and then centrifuged at 8000 rpm for 10 minutes, a supernatant was collected, and subjected to 3000 kDa ultrafiltration and freeze-drying, so as to obtain the PLGA nanoparticles in which the drug was embedded, and the PLGA nanoparticles were redissolved with normal saline. In order to prevent the problem of poor dispersion performance caused by adhesion between the microspheres after freeze-drying and redissolution of the microspheres, a freeze-drying protective agent was also added before freeze-drying. In this embodiment, the freeze-drying protective agent was a PVA solution, and a mass concentration of the PVA solution was 1% (w/v).

An average particle size of the PLGA nanospheres in which the drugs were embedded obtained in this embodiment was 400 nm.

In S3, the macrophage membrane and the c-RGD peptide were wrapped outside the PLGA nanospheres containing the drug by a membrane pushing method:

the macrophage membrane, the c-RGD peptide and the PLGA nanospheres in which the drug molecules were embedded were uniformly mixed, and the obtained mixture was extruded by an extruder (Sigma-Aldrich, SKU: 610000-1EA), so that the mixture was extruded through polycarbonate membranes of 800 nm, 400 nm and 200 nm respectively (5 to 10 times for each membrane), and the targeted traditional Chinese medicine in-situ tumor vaccine was obtained after extrusion.

A particle size of the targeted traditional Chinese medicine in-situ tumor vaccine was less than or equal to 200 nm.

The targeted traditional Chinese medicine in-situ tumor vaccine provided by this embodiment could realize targeted slow-release administration for pancreatic cancer in vivo, and could achieve the effects of accurate administration and long circulation of the drug at the tumor site. The preparation method of the targeted traditional Chinese medicine in-situ tumor vaccine was simple and highly flexible, and the tumor vaccine was suitable for carrying a drug for treating pancreatic cancer, especially a drug for treating pancreatic cancer through immunotherapy.

### Embodiment 3 Preparation of Targeted Traditional Chinese Medicine In-Situ Tumor Vaccine without Wrapping by c-RGD Peptide

This embodiment was the same as Embodiment 2, and the difference was in the step S3 that the macrophage membrane was wrapped outside the PLGA nanospheres containing the drug by the membrane pushing method.

### Embodiment 4 Preparation of Targeted Traditional Chinese Medicine In-Situ Tumor Vaccine without Wrapping by Macrophage Membrane

This embodiment was the same as Embodiment 2, and the difference was in the step S3 that the c-RGD peptide was wrapped outside the PLGA nanospheres containing the drug by the membrane pushing method.

### Embodiment 5 Preparation of Targeted Traditional Chinese Medicine In-Situ Tumor Vaccine

This embodiment was the same as Embodiment 2, and the difference was in the step S22 that only 0.3 mg of the Brusatol was wrapped without adding the Lycium barbarum polysaccharide.

### Embodiment 6 Detection of Physical and Chemical Properties Based on Targeted Traditional Chinese Medicine In-Situ Tumor Vaccine

The targeted traditional Chinese medicine in-situ tumor vaccine prepared by Embodiment 2 was subjected to stability detection, encapsulation efficiency detection and release rate detection respectively.

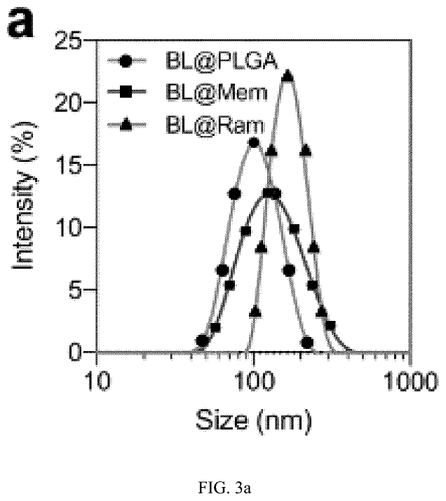

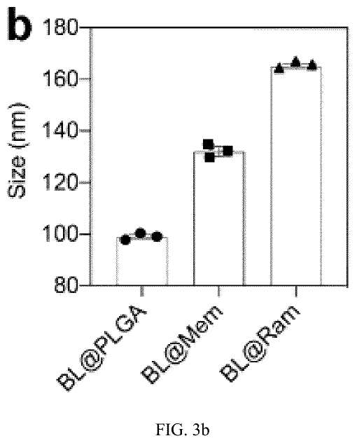

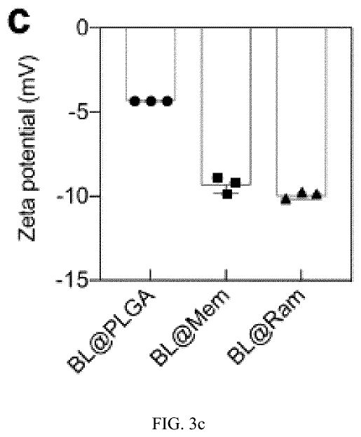

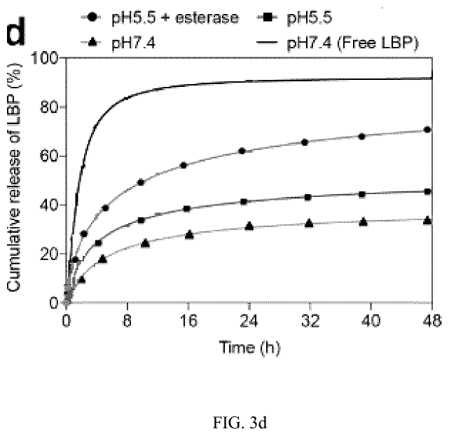

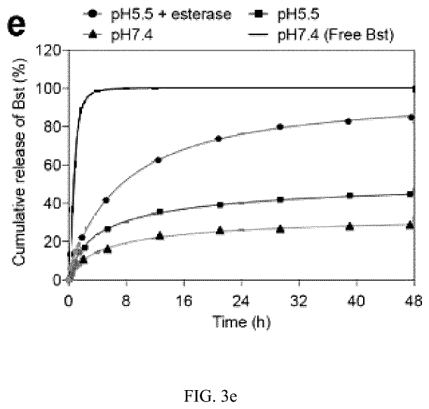

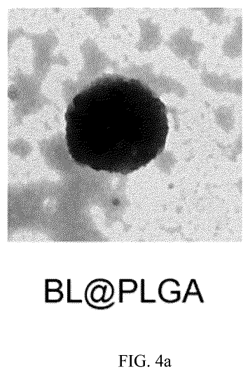

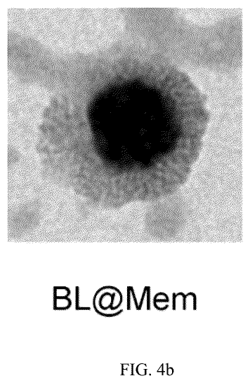

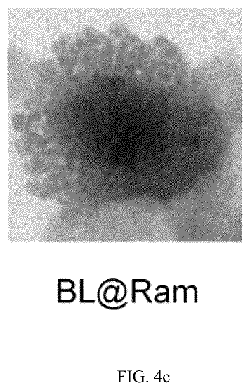

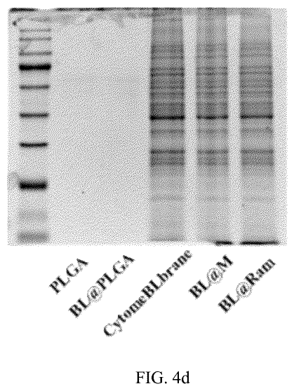

The targeted traditional Chinese medicine in-situ tumor vaccine prepared by Embodiment 2 was subjected to stability detection: The tumor vaccine was dispersed in an RPMI 1640 culture medium containing 10% serum, and the stability of the tumor vaccine was judged by changes of particle size, potential and particle size dispersion index of the targeted traditional Chinese medicine in-situ tumor at different time points. Details were shown in FIG. 4. It could be seen that the particle size, the potential and the particle size dispersion index of the targeted traditional Chinese medicine in-situ tumor vaccine prepared by Embodiment 2 were stable in the RPMI 1640 culture medium containing 10% serum. Details were shown in a, b and c in FIG. 3. It was proved that the targeted traditional Chinese medicine in-situ tumor vaccine prepared by Embodiment 2 had good stability.

The targeted traditional Chinese medicine in-situ tumor vaccine prepared by Embodiment 2 was subjected to encapsulation efficiency detection: A liquid obtained after ultrafiltration of the targeted traditional Chinese medicine in-situ tumor vaccine obtained in the S23 in Embodiment 2 was collected, and a content of drug not wrapped in the targeted traditional Chinese medicine in-situ tumor vaccine was determined. The Brusatol was subjected to gradient elution by high performance liquid chromatography (HPLC), wherein a mobile phase was acetonitrile-water, and the gradient elution was carried out with 5% to 30% acetonitrile for 0 to 4 minutes, and with 30% to 36% acetonitrile for 4 minutes to 30 minutes; a detection wavelength was 280 nm; a column temperature was 25° C.; a flow rate was 1 mL/min; a separation column was Cis, and appearance time was 18 minutes. Details were shown in c in FIG. 4. The Lycium barbarum polysaccharide was determined by a phenol-sulfuric acid method: A liquid after ultrafiltration was taken, added with 1.0 mL of distilled water, then added with 1.0 mL of 6% phenol, and quickly added with 5.0 mL of concentrated sulfuric acid, the mixture was shaken on a vortex instrument to be fully mixed, and allowed to stand for 30 minutes, and then an absorbance at 490 nm was determined. Details were shown in d in FIG. 4. Results showed that, in the targeted traditional Chinese medicine in-situ tumor vaccine, encapsulation efficiency of the Brusatol was 70±0.85%, and encapsulation efficiency of the polysaccharide was 93.7±2.21%.

The targeted traditional Chinese medicine in-situ tumor vaccine prepared by Embodiment 2 was subjected to release rate detection: The prepared targeted nanospheres were redissolved and then placed in a dialysis bag, the dialysis bag was placed in centrifuge tubes added with liquids at different pH values and placed in a shaker (37° C., 1000 rpm), the liquids in the centrifuge tubes were taken at multiple time points, and replenishment was carried out for the taken liquids. Contents of Brusatol and Lycium barbarum polysaccharide in the taken liquids were detected by the above high performance liquid chromatography and phenol sulfuric acid method respectively. Details were shown in d and e in FIG. 3. Release rates of the Lycium barbarum polysaccharide and the Brusatol in different environments within 48 hours were all as follows: ph 5.5+esterase>pH 5.5>pH 7.4, which proved that the targeted traditional Chinese medicine in-situ tumor vaccine was released rapidly in simulated tumor environment.

### Embodiment 7 Detection of Micromorphologic Composition of Targeted Traditional Chinese Medicine In-Situ Tumor Vaccine

The targeted traditional Chinese medicine in-situ tumor vaccine prepared by Embodiment 2 was detected by a transmission electron microscope and sodium dodecyl sulfate-polyacrylamide gel electrophoresis.

The targeted traditional Chinese medicine in-situ tumor vaccine prepared by Embodiment 2 was detected by the transmission electron microscope: The targeted nanospheres prepared by Embodiment 2 were dropwise added onto a copper net, negatively stained with uranium acetate, and observed by the transmission electron microscope (JEM-2100F) at 120 kV. Details could be observed from a, b and c in FIG. 4 (a: A tumor vaccine wrapped by the c-rgd and the macrophage membrane; b: A tumor vaccine wrapped by the macrophage membrane; and c: A tumor vaccine without wrapping by the c-rgd and the macrophage membrane). It could be observed from results that the tumor vaccine wrapped by the macrophage membrane had an obvious membrane trace and a larger volume compared with the tumor vaccine without wrapping by the macrophage membrane, which proved that the targeted traditional Chinese medicine in-situ tumor vaccine was wrapped by the macrophage membrane on surface.

The targeted traditional Chinese medicine in-situ tumor vaccine prepared by Embodiment 2 was detected by the sodium dodecyl sulfate-polyacrylamide gel electrophoresis: The targeted traditional Chinese medicine in-situ tumor vaccine prepared by Embodiment 2 was subjected to the sodium dodecyl sulfate-polyacrylamide gel electrophoresis, and whether there was a membrane protein on the surface of the targeted traditional Chinese medicine in-situ tumor vaccine was observed. Details were shown in d in FIG. 4. It could be seen that a SDS-PAGE gel of the tumor vaccine with the macrophage membrane on surface was consistent with the electrophoresis run by the macrophage membrane, which indicated that there was the membrane protein on the surface of the targeted traditional Chinese medicine in-situ tumor vaccine.

### Embodiment 8 Detection of Uptake Situations of Macrophages and Pancreatic Cancer Cells on Targeted Traditional Chinese Medicine In-Situ Tumor Vaccine

The targeted traditional Chinese medicine in-situ tumor vaccines prepared by Embodiment 2, Embodiment 3 and Embodiment 4 were subjected to an uptake experiment of pancreatic cancer cells.

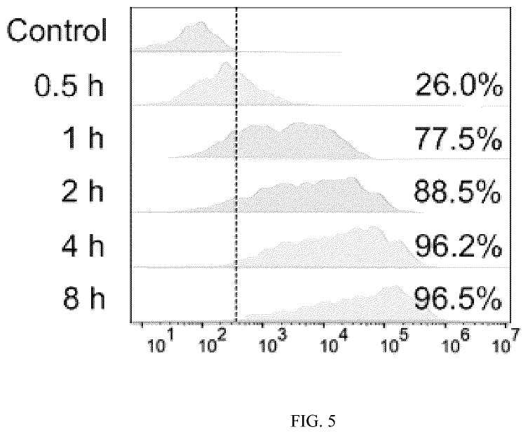

PANC-02 pancreatic cancer cells were cultured, and the targeted traditional Chinese medicine in-situ tumor vaccines prepared by Embodiment 2, Embodiment 3 and Embodiment 4 in which a fluorescent probe DiD was wrapped were added into the culture medium of the PANC-02 pancreatic cancer cells. Uptake situations of the PANC-02 pancreatic cancer cells to the targeted traditional Chinese medicine in-situ tumor vaccines were observed by flow cytometry, and results were shown in FIG. 5, which showed that the PANC-02 pancreatic cancer cells could basically complete the uptake of the targeted traditional Chinese medicine in-situ tumor vaccines within 4 hours. The tumor vaccines prepared by Embodiment 3 and Embodiment 4 were subjected to the same experiment, and results showed that uptake time of the PANC-02 pancreatic cancer cells to the tumor vaccines was longer than that of the PANC-02 pancreatic cancer cells to the tumor vaccine prepared by Embodiment 2.

### Embodiment 9 Killing Effect of Targeted Traditional Chinese Medicine In-Situ Tumor Vaccine on Pancreatic Cancer Cells and Activation Situation of Targeted Traditional Chinese Medicine In-Situ Tumor Vaccine on Immune Cells

The targeted traditional Chinese medicine in-situ tumor vaccine prepared by Embodiment 2 was subjected to activation detection of dendritic cells.

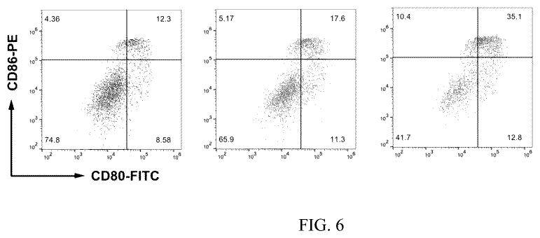

The targeted traditional Chinese medicine in-situ tumor vaccine prepared by Embodiment 2 was subjected to the activation detection of dendritic cells: BMDC was extracted from mouse bone marrow for culture, the targeted traditional Chinese medicine in-situ tumor vaccine prepared was given to the pancreatic cancer cells, two days later, a supernatant was extracted and added into the BMDC, and expressions of CD80 and CD86 in the BMDC were detected by flow cytometry. Details were shown in FIG. 6. Compared with the control group, CD80 and CD86 in mice injected with the targeted traditional Chinese medicine in-situ tumor vaccine were increased significantly, which indicated that the BMDC was activated.

### Embodiment 10 Treatment Situation of Targeted Traditional Chinese Medicine In-Situ Tumor Vaccine on Tumor-Bearing Mice with Pancreatic Cancer

The targeted traditional Chinese medicine in-situ tumor vaccines prepared by Embodiment 2 and Embodiment 5 were subjected to an animal experiment to detect a tumor change and a tumor targeting ability of tumor-bearing mice.

The targeted nanospheres prepared by Embodiment 2 and Embodiment 5 were subjected to detection of tumor size change of the tumor-bearing mice.

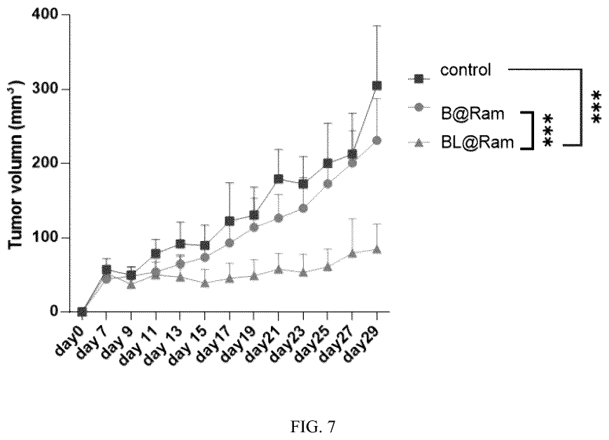

C57BL/6J mice (4 weeks old) were selected, and each mouse was inoculated with 1*10⁶ PANC-02 pancreatic cancer cells. One week later, the targeted traditional Chinese medicine in-situ tumor vaccine was injected into a tail vein, once every two days, for a total of three times, a change of a tumor size was recorded every two days, and the mice were killed after 30 days of inoculation. Details were shown in FIG. 7. After 30 days of inoculation, a tumor volume of mice injected with the targeted traditional Chinese medicine in-situ tumor vaccine of Embodiment 2 (red) was smaller than that of mice injected with the targeted traditional Chinese medicine in-situ tumor vaccine of Embodiment 5 (blue), which indicated that the combination of Brusatol and Lycium barbarum polysaccharide was superior to single Brusatol, and could achieve a better synergistic anti-tumor effect.

What is claimed is:

1. A targeted traditional Chinese medicine in-situ tumor vaccine, comprising two drugs, PLGA nanospheres and a surface modification system, wherein the drug is embedded into the PLGA nanospheres; the drugs are Lycium barbarum polysaccharide and Brusatol; and the surface modification system is a macrophage membrane and c-RGD peptide modification system.

2. The targeted traditional Chinese medicine in-situ tumor vaccine according to claim 1, wherein a mass ratio of the PLGA nanospheres to the macrophage membrane is 5:1 to 10:1, a mass ratio of the PLGA nanospheres to the c-RGD peptide is 15:1 to 20:1, a mass ratio of the PLGA nanospheres to the Lycium barbarum polysaccharide is 4:1 to 8:1, and a mass ratio of the PLGA nanospheres to the Brusatol is 20:1 to 30:1.

3. The targeted traditional Chinese medicine in-situ tumor vaccine according to claim 1, wherein a particle size of the PLGA nanospheres is less than or equal to 200 nm.

4. A preparation method of the targeted traditional Chinese medicine in-situ tumor vaccine according to claim 1, comprising the following steps:
S1: Extracting the macrophage membrane: Lysing macrophages by a cell lysis buffer, and then extracting the cell membrane by a layered high-speed centrifugation method for standby application;
S2: Preparing the PLGA nanospheres in which the drug is embedded: Embedding the Lycium barbarum polysaccharide and the Brusatol into the PLGA nanospheres by a double emulsification method; and
S3: Wrapping the PLGA nanospheres in which the drug is embedded into the surface modification system by a membrane extrusion method.

5. The preparation method according to claim 4, wherein the double emulsification method in the step S2 comprises: Dissolving the PLGA in an acetone organic solvent to obtain a premix; dissolving the Brusatol in dimethyl sulfoxide, dissolving the Lycium barbarum polysaccharide in normal saline, adding the solutions into the premix under stirring, and subjecting the mixture to an ice bath ultrasonic treatment to form a water-oil emulsion; uniformly mixing the water-oil emulsion with a solution containing poloxamer 188 to obtain a water-oil-water emulsion; and subjecting the water-oil-water emulsion to an ice bath ultrasonic treatment or a homogenization treatment, then removing excessive acetone by volatilization, ultra-freezing, centrifuging and washing the emulsion, and then freeze-drying the emulsion to obtain the PLGA nanospheres in which drug molecules are embedded.

6. The preparation method according to claim 4, wherein the membrane extrusion method in the step S3 comprises: Mixing the surface modification system with the PLGA nanospheres in which the drug is embedded, and then subjecting the mixture to an extrusion treatment by an extruder, wherein a polycarbonate membrane is used as a filter structure in a process of the extrusion treatment.

7. The preparation method according to claim 5, wherein concentrations of the Lycium barbarum polysaccharide and the Brusatol are 1.5 mg/mL and 0.15 mg/mL respectively.

8. The preparation method according to claim 5, wherein before freeze-drying, the method further comprises a step of adding a freeze-drying protective agent, wherein the freeze-drying protective agent is polyvinyl alcohol or mannitol, and a mass concentration of the freeze-drying protective agent is 0.5% to 1%.

9. A method for treating a cancer comprising a step of administering a subject in need with the targeted traditional Chinese medicine in-situ tumor vaccine of claim 1.

10. The method according to claim 9, wherein the cancer is a pancreatic cancer.
